(19) United States
(12) Patent Application Publication
(10) Pub. No.: US 2023/0268076 A1
(43) Pub. Date: Aug. 24, 2023

(54) PREDICTIVE ANALYSIS OF PREDICTED MICROVASCULAR OUTCOMES USING PREDICTIVE MODELS

(71) Applicant: Optum Services (Ireland) Limited, Dublin (IE)

(72) Inventors: Kevin A. HEATH, Dublin (IE); Kieran O'DONOGHUE, Dublin (IE); Paul J. GODDEN, London (GB); Terry J. SCHERR, Phoenix, AZ (US)

(21) Appl. No.: 18/051,652

(22) Filed: Nov. 1, 2022

Related U.S. Application Data
(60) Provisional application No. 63/268,316, filed on Feb. 22, 2022.

(51) Int. Cl. G16H 50/30; G16H 50/50

(52) U.S. Cl. CPC G16H 50/30; G16H 50/50

(57) ABSTRACT

There is a need for more effective and efficient health risk assessment and prevention. This need is addressed by utilizing microvascular information as a component in an integrated health risk score. An example method can include receiving a microvascular data object for a user that is indicative of a microvascular volume differential for the user; determining a plurality of predictive outcome features for a predicted microvascular outcome; determining, using a feature prediction model, one or more impact predictions for the plurality of predictive outcome features; generating, based at least in part on the microvascular data object and the one or more impact predictions, an outcome risk score data object for the user; and generating, using a refined remedial model, and based at least in part, on the outcome risk score data object, at least one tailored remedial measure for the user.

### CROSS-REFERENCE TO RELATED APPLICATIONS

This application claims the benefit of U.S. Provisional Application No. 63/268,316, entitled “Risk Assessment Using Volume Plethysmography for Prognosis and Mitigation of Adverse Events,” and filed Feb. 22, 2022, the entire contents of which are hereby incorporated by reference.

### BACKGROUND

Various embodiments of the present invention address technical challenges related to health risk assessment and management such as, for example, to prevent adverse microvascular events and/or diseases. Various embodiments of the present invention address the shortcomings of conventional health risk assessment techniques and disclose various techniques for reliably and accurately generating health risk assessments for short term intervals using microvascular data.

### BRIEF SUMMARY

In general, embodiments of the present invention provide methods, apparatus, systems, computing devices, computing entities, and/or the like for predictive analysis of predicted microvascular outcomes using predictive models. Certain embodiments of the present invention utilize systems, methods, and computer program products that implement a predictive model for: (i) determining a predicted microvascular outcome risk for a user based at least in part on microvascular information and (ii) generating remedial measures for the mitigation of the predicted microvascular outcome risk.

In accordance with one aspect, a method for predicting predicted microvascular outcomes using microvascular data objects is provided. The computer-implemented method comprises receiving, by one or more processors, a microvascular data object for a user. The microvascular data object is indicative of a microvascular volume differential for the user. The method comprises determining, by the one or more processors, a plurality of predictive outcome features for a predicted microvascular outcome. The method comprise determining, by the one or more processors and using a feature prediction model, one or more impact predictions for the plurality of predictive outcome features. The method comprises generating, by the one or more processors and based at least in part on the microvascular data object and the one or more impact predictions, an outcome risk score data object for the user. The method comprises generating, by the one or more processors, using a refined remedial model and based at least in part on the outcome risk score data object, at least one tailored remedial measure for the user.

In accordance with another aspect, an apparatus for predicting predicted microvascular outcomes using microvascular data objects is provided. The apparatus comprises at least one processor and at least one memory including program code. The at least one memory and the program code are configured to, with the processor, cause the apparatus to at least receive a microvascular data object for a user. The microvascular data object is indicative of a microvascular volume differential for the user. The apparatus is further caused to determine a plurality of predictive outcome features for a predicted microvascular outcome. The apparatus is further caused to determine, using a feature prediction model, one or more impact predictions for the plurality of predictive outcome features. The apparatus is further caused to generate, based at least in part on the microvascular data object and the one or more impact predictions, an outcome risk score data object for the user. The apparatus is further caused to generate, using a refined remedial model and based at least in part on the outcome risk score data object, at least one tailored remedial measure for the user.

In accordance with yet another aspect, a computer program product for predicting predicted microvascular outcomes using microvascular data objects is provided. The computer program product comprises at least one non-transitory computer-readable storage medium having computer-readable program code portions stored therein. The computer-readable program code portions are configured to receive, by one or more processors, a microvascular data object for a user. The microvascular data object is indicative of a microvascular volume differential for the user. The computer-readable program code portions are configured to determine, by one or more processors, a plurality of predictive outcome features for a predicted microvascular outcome. The computer-readable program code portions are configured to determine, by the one or more processors and using a feature prediction model, one or more impact predictions for the plurality of predictive outcome features. The computer-readable program code portions are configured to generate, by the one or more processors and based at least in part on the microvascular data object and the one or more impact predictions, an outcome risk score data object for the user. The computer-readable program code portions are configured to generate, by the one or more processors, using a refined remedial model and based at least in part on the outcome risk score data object, at least one tailored remedial measure for the user.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example external computing entity in accordance with some embodiments discussed herein.

FIG. 4 is a flowchart diagram of an example process for generating an outcome risk score for a predicted microvascular outcome in accordance with some embodiments discussed herein.

FIG. 5 is a flowchart diagram of an example process for determining that the microvascular data object achieves the risk criteria in accordance with some embodiments discussed herein.

FIG. 6 is a flowchart diagram of an example process for refining an outcome risk score for a user in accordance with some embodiments discussed herein.

FIG. 7 is a flowchart diagram of an example process for generating an outcome risk score data object for a user based at least in part on a historical microvascular data object in accordance with some embodiments discussed herein.

FIG. 8 is a flowchart diagram of an example process for automatically generating a tailored remedial measure for a user based at least in part on a microvascular data object in accordance with some embodiments discussed herein.

FIG. 9 is an operational example of an interactive user interface for a user in accordance with some embodiments discussed herein.

### DETAILED DESCRIPTION

Various embodiments of the present invention now will be described more fully herein with reference to the accompanying drawings, in which some, but not all, embodiments of the inventions are shown. Indeed, these inventions can be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to textual data analysis, one of ordinary skill in the art will recognize that the disclosed concepts can be used to perform other types of data analysis.

### I. Overview

Aspects of the present invention present predictive techniques for determining and/or mitigating risk of adverse predicted microvascular outcomes such as, for example, cardiovascular diseases or events. The predictive techniques utilize microvascular information obtained through non-invasive, easily accessible, volume plethysmography techniques to create an integrated risk score that evaluates a user's risk of a predicted microvascular outcome regardless of previous vascular diagnoses.

By incorporating microvascular information to an integrated risk score, the predictive techniques of the present disclosure provides more accurate and shorter-term risk predictions for predicted microvascular outcomes than conventional techniques. For instance, conventional risk assessment techniques rely on known risk factors including age, sex, race, blood pressure, lipid profile, diabetic, and smoking history, and interventions such as blood pressure treatment, statin, and aspirin use. However, the individual impact of each risk factor is not measurable due to the lack of a direct assessment of overall health. The predictive techniques of the present disclosure utilizes microvascular information indicative of the blood flow through a user's microvasculature to address this unmet challenge by representing the user's overall health using peripheral vasculature disease status. This can improve health risk assessment for symptomatic users and, unlike previous techniques, can detect health risks in asymptomatic users with no prior symptoms or events.

The integrated risk score of the present disclosure can be utilized to independently predict risk of adverse predicted microvascular outcomes. These independent predictions can be observed over time to generate a feature prediction model that correlates predicted microvascular outcome features with adverse predicted microvascular outcomes. For example, the health risk assessment method can utilize microvascular information as a proxy for overall health. A user's overall health can be monitored over time and compared to known predictive outcome features for predicted microvascular outcomes. In this manner, a predictive outcome feature's impact on predicted microvascular outcomes can be measured and leveraged to provide tailored remedial measures for mitigating a user's risk of adverse predicted microvascular outcomes. Microvascular information can be continuously collected overtime to refine remedial measures based at least in part on insights derived thereof.

Exemplary inventive and technologically advantageous aspects of the present invention include: (i) the use of volume plethysmography techniques to identify health risks for a user; (ii) using microvascular information as a direct measure of overall health; and (iii) incorporating the microvascular information into an integrated risk score that is valid for asymptomatic individuals with no prior vascular diagnosis at time intervals of one and three years. In addition, the inventive and technologically advantageous aspects of the present invention include the use of microvascular information in a feature prediction model that allows for targeted predicted microvascular outcome mitigation treatments based at least in part on observed impacts of individual predictive outcome features on a user's health. By allowing the measurement of the impact of individual predictive outcome features to the health of a user, the microvascular data described herein can enable targeted remedial measures that provide alternative “next best action” options for risk mitigation. These options can be presented in a novel interactive user interface that (i) is tailored to a user's individual factors and preferences, (ii) is reconfigurable based at least in part on user input, and (iii) that automatically updates based at least in part on real world data.

### II. Definitions

The term “microvascular data object” can refer to a data entity that is configured to describe a microvascular blood flow in one or more regions of a user's body. The microvascular data object, for example, can be indicative of a microvascular volume differential for a user. The microvascular volume differential can represent one or more flow abnormalities in one or more regions of a user's body. In some embodiments, the microvascular volume differential for a user can be based at least in part on oxygen saturation levels in one or more different microvascular regions of a user's body. A microvascular volume differential can be indicative of a presence and/or severity of PAD including, for example, LE-PAD. PAD and LE-PAD, for instance, can be identified by one or more flow abnormalities at one or more different microvascular regions of a user's body.

The microvascular data object can include a parameter representative of a microvascular blood flow for the user at a point in time. For instance, the parameter can include one or more microvascular health measurements. A microvascular health measurement can include a microvascular health value for a user that represents the user's microvascular blood flow. The microvascular health value can be represented by a percentage, fraction, and/or any other numerical representation for representing a proportion of a user's microvascular blood flow. As examples, a microvascular health value of 0.9 (e.g., 90%, 9/10, etc.) can indicate a high (e.g., healthy) blood flow rate for a user, whereas a microvascular health value of 0.4 (e.g., 40%, 4/10, etc.) can indicate a low (e.g., unhealthy) blood flow rate for a user. As described herein, a microvascular health value can be representative of a user's overall health. Thus, the microvascular health value can be used as a proxy for a user's risk for predicted microvascular outcomes.

The term “historical microvascular data object” can refer to a data entity that is configured to describe a plurality of historical microvascular health measurements for a plurality of users over time. For example, the plurality of historical microvascular health measurements for the plurality of users over time can include a plurality of microvascular health values computed for each of the plurality of users. The plurality of historical microvascular health measurements can be measured using volume-based measurement techniques during one or more historical clinical visits, house calls, etc. in which a user's health can be observed. In some embodiments, the volume-based measurement techniques can be used by a user to self-diagnosis the user's health.

The historical microvascular data object can include a historical log of a plurality of historical microvascular health measurements for each of the plurality of users. Each historical microvascular health measurement can be recorded and associated with a particular user at a particular time. In some embodiments, the microvascular health measurements can be recorded in a time-series model that correlates the historical microvascular health measurements with one or more predicted microvascular outcome features over time. In some embodiments, the plurality of historical microvascular health measurements can be stored in association with a user profile data object.

The term “user profile data object” can refer to a data entity that is configured to describe user information for a particular user. A user can include an individual person such as a patient, an employee, and/or the like. The user information can include user characteristics, associated predictive outcome features, microvascular health measurements, and/or any other information for the determination and/or mitigation of predicted microvascular outcomes for the particular user.

For example, the user profile data object can describe one or more user characteristics for a user including, as examples, a user medical history (e.g., allergies, historical clinical procedures/recommendations, etc.), demographic information (e.g., social determinants, etc.), preferences (e.g., habits such as smoking, diagnosis preferences, etc.), and/or the like.

As another example, the user profile data object can describe a historical and/or current overall health for the particular user. A user's overall health, for example, can be represented by a microvascular health measurement for the user. The user profile data object can describe a plurality of historical and/or current microvascular health measurements for the user. In some embodiments, the user profile data object can include an analysis of one or more trends and/or other secondary insights associated with the historical and/or current microvascular health measurements for the user.

As yet another example, the user profile data object can describe one or more predictive outcome features associated with the user. In some embodiments, the one or more predictive outcome features described by the user profile data object can correspond to user characteristics for the user. By way of example, the associated predictive outcome features can include historical clinical procedures, social determinants, smoking habits and/or any other characteristics of a particular user.

The term “predictive outcome feature” can refer to a risk factor associated with a predicted microvascular outcome. Example risk factors can include smoking, lack of exercise, diet, obesity, high blood pressure, high cholesterol, medical history, age, and/or any other characteristic with a demonstrated association or correlation to predicted microvascular outcomes.

The predictive outcome features for a predicted microvascular outcome can include a plurality of general risk factors that are applicable to a plurality of different predicted microvascular outcomes or specific risk factors that are applicable to a specific predicted microvascular outcome. The plurality of predictive outcome features can be received from a plurality of different clinical sources. By way of example, clinical literature and/or expertise can be leveraged to source a list of clinically relevant risk factors for predicted microvascular outcomes such as the predicted microvascular outcome of interest. In some embodiments, the plurality of predictive outcome features can be augmented by secondary factors that may contribute to the likelihood of a predicted microvascular outcome. Secondary factors, for example, can include social determinants of health as identified via deprivation index, etc. By way of example, areas of high deprivation can lead to increased risk for predicted microvascular outcomes.

The term “volume-based measurement techniques” can refer to systems and methods for obtaining a microvascular data object. The volume-based measurement techniques can include volume plethysmography (VP) techniques. The VP techniques, for example, can obtain microvascular health measurements using VP systems for measuring microvascular blood flow volumes at one or more microvascular regions of a user. The VP techniques can include any number of different VP systems capable of measuring microvascular flow volumes at one or more different microvascular regions of a user's body. One exemplary VP system, for example, can include a peripheral artery disease screen such as, for example, QuantaFlo™ in which a sensor is placed on a user's left toe, right toe, left finger, and right finger for a period of time (e.g., fifteen seconds) to determine blood flow in each of the user's legs and arms. The blood flow data can be analyzed to determine a microvascular health measurement that is indicative of microvascular blood pressure between upper and lower extremities of a user.

Unlike macrovascular measurement systems such as, for example, ankle-brachial index screenings which utilize large vessel (macrovascular) blood pressure measurements between upper and lower extremities, VP systems can assess microvascular flow volumes that provide a more accurate depiction of a particular individual's health. The VP techniques can include a non-invasive screening that can be easily performed in the primary care setting and is widely scalable to improve the assessment and management of health risk. VP techniques have been demonstrated to independently predict predicted microvascular outcomes including hospitalization for myocardial infarction and stroke at one- and three-years post-screening. While VP techniques are used for the diagnosis of certain diseases such LE-PAD, conventional cardiovascular screening techniques do not use VP techniques to estimate health risk.

The term “predicted microvascular outcome” can refer to any disease and/or damage to any organ system sensitive to micro- or macro- vascular flow. The predicted microvascular outcome can include a microvascular disease impacting any organ system including the brain, retina, kidney, lung, heart, etc. For instance, microvascular disease can include a varied set of conditions that can ultimately result in tissue damage and progressive organ failure. Example manifestations of microvascular disease include the development and/or progression of skin ulcers, minor amputations, chronic kidney disease, proteinuria, peripheral neuropathy, retinopathy, blindness, pulmonary arterial hypertension, ischemic heart disease, heart failure, and/or the like.

In some cases, the predicted microvascular outcome can include cardiovascular disease and/or event. A cardiovascular disease, for example, can include any condition that can impact a user's heart or blood vessels. By way of example, a cardiovascular disease can include peripheral polyneuropathy, chronic kidney disease, atherosclerotic cardiovascular disease, coronary artery disease, high blood pressure, congestive heart failure, arrhythmias, PAD, LE-PAD, congenital heart disease, and/or any other condition that can impact the performance of a user's cardiovascular system. A cardiovascular event, for example, can include any incident that may cause damage to a user's heart muscle. By way of example, a cardiovascular event can include MACE including strokes, myocardial infarction, cardiovascular death and/or any other event that can impact the performance of a user's cardiovascular system.

The term “outcome risk score data object” can refer to a data entity that is configured to describe a particular individual's risk for a predicted microvascular outcome within a time period. For example, the outcome risk score data object for a user can be indicative of a short-term likelihood (e.g., one-to-three-year time span) that a user will develop a microvascular disease and/or a short-term likelihood (e.g., one-to-three-year time span) of an occurrence of a microvascular event. In addition, or alternatively, the outcome risk score data object for a user can be indicative of a long-term likelihood (e.g., five-year or longer time span) that a user will develop a microvascular disease and/or a long-term likelihood (e.g., e.g., five-year or longer time span) of an occurrence of a microvascular event. The outcome risk score data object can be descriptive of an overall outcome risk score and/or a specific outcome risk score.

The term “overall outcome risk score” can refer to a particular individual's overall risk for any predicted microvascular outcome. The overall outcome risk score can be associated with a plurality of predicted microvascular outcomes. For instance, the overall outcome risk score can be descriptive of a user's overall health. In some embodiments, a current microvascular health value can be used as a proxy for an overall outcome risk score.

The term “specific outcome risk score” can refer to a particular individual's specific risk for a predicted microvascular outcome of interest. The specific outcome risk score can be descriptive of a likelihood that a user will develop a specific microvascular disease and/or suffer an occurrence of a specific microvascular event. In some embodiments, a current microvascular health value can be used as a proxy for a specific outcome risk score and refined using one or more predictive outcome features associated with the predicted microvascular outcome of interest.

The term “feature prediction model” can refer to a computer-implemented data structure configured to identify one or more impact predictions for a plurality of predictive outcome features of a predicted microvascular outcome. For instance, the feature prediction model can correlate the historical microvascular data object and the plurality of predictive outcome features over time. The feature prediction model can be based at least in part on the historical microvascular data object. For instance, the feature prediction model can be configured to predict one or more impact predictions for the plurality of predictive outcome features based at least in part on one or more mapped relationships between the historical microvascular data object and the plurality of predictive outcome features over time.

As one example, the feature prediction model can include a time-series model that is descriptive of a plurality of historical microvascular health measurements and a plurality of predictive outcome features associated with each of the plurality of historical microvascular health measurements over time. The time-series model can be generated by tabulating data sources for predictive modeling of the plurality of predictive outcome features against predicted microvascular outcomes. The information can be collated in a time series format versus predicted microvascular outcomes for the plurality of users. The time series format can utilize any time series framework that can be applied for modeling purposes. In this manner, a feature prediction model can be generated that includes a plurality of microvascular health measurements that are recorded as a function of time in correlation with one or more predictive outcome features over time. By way of example, the feature prediction model can employ social determinants of health as one component, a direct measure of microvasculature as another component, a clinical measure as a third, etc.

As another example, the feature prediction model can include a machine-learning based model. The feature prediction model can include any type of machine-learning based model including one or more supervised, unsupervised, and/or reinforcement learning models. In some implementations, the feature prediction model can include a first machine-learning based prediction model configured to learn one or more associations between the plurality of predictive outcome features and a predicted microvascular outcome based at least in part on microvascular information. By way of example, the feature prediction model can include one or more neural networks (e.g., feedforward artificial neural networks, perceptron and multilayer perceptron neural networks, radial basis function artificial neural networks, recurrent neural networks, modular neural networks, etc.) trained using one or more supervised, unsupervised, and/or reinforcement training techniques based at least in part on a historical microvascular data object.

The term “refined remedial model” can refer to a computer-implemented data structure configured to quantify a potential impact of at least one of the plurality of predictive outcome features on an outcome risk score data object. By way of example, the refined remedial model can include a causal propensity model configured to quantify statistically the impact of predictive outcome features upon real-world change (e.g., as represented by microvascular health measurements).

As another example, the refined remedial model can include a machine-learning based model. The refined remedial model can include any type of machine-learning based model including one or more supervised, unsupervised, and/or reinforcement learning models. In some implementations, the refined remedial model can include a second machine-learning based prediction model configured to learn one or more associations between the plurality of predictive outcome features and a microvascular data object for a user based at least in part on microvascular information for a plurality of users. By way of example, the refined remedial model can include one or more neural networks (e.g., feedforward artificial neural networks, perceptron and multilayer perceptron neural networks, radial basis function artificial neural networks, recurrent neural networks, modular neural networks, etc.) trained using one or more supervised, unsupervised, and/or reinforcement training techniques based at least in part on a historical microvascular data object.

The term “tailored remedial measure data object” can refer to a data entity that is configured to describe a remedial measure for a user to mitigate a predicted microvascular outcome. The remedial measure for the user can be based at least in part on the microvascular data object and the outcome risk score data object for the user. The tailored remedial measure can be associated with at least one of the plurality of predictive outcome features. For example, in response to a negative outcome risk score data object and a corresponding microvascular data object, the tailored remedial measure can identify one or more predictive outcome features associated with the user that may be modified through treatments or altered behavior.

The tailored remedial measure data object can include a plurality of alternative treatments and an indication of an expected impact to the outcome risk score associated with at least one of the plurality of alternative treatments. For example, the tailored remedial measure data object can provide a plurality of different options for managing an outcome risk score data object. Each option can identify (i) a treatment and/or altered behavior associated with a predictive outcome feature and (ii) a potential impact to the outcome risk score data object based at least in part on the treatment and/or altered behavior. As a consequence, the tailored remedial measure data object can include a “next-best option” for a given user in the event one option is preferred over another (e.g., a user refuses to quit smoking, but an increase to their statin results in similar risk mitigation).

In some implementations, the tailored remedial measure data object can be based at least in part on user data. For example, a predictive data analysis computing entity can receive a user profile data object descriptive of user information for the user. As one example, the user profile data object can include one or more user preferences. In some implementations, the plurality of different options for managing the outcome risk score data object can be selected, ordered, and/or presented based at least in part on one or more user preferences.

### III. Computer Program Products, Methods, and Computing Entities

Embodiments of the present invention can be implemented in various ways, including as computer program products that comprise articles of manufacture. Such computer program products can include one or more software components including, for example, software objects, methods, data structures, or the like. A software component can be coded in any of a variety of programming languages. An illustrative programming language can be a lower-level programming language such as an assembly language associated with a particular hardware architecture and/or operating system platform. A software component comprising assembly language instructions can require conversion into executable machine code by an assembler prior to execution by the hardware architecture and/or platform. Another example programming language can be a higher-level programming language that can be portable across multiple architectures. A software component comprising higher-level programming language instructions can require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages can be executed directly by an operating system or other software component without having to be first transformed into another form. A software component can be stored as a file or other data storage construct. Software components of a similar type or functionally related can be stored together such as, for example, in a particular directory, folder, or library. Software components can be static (e.g., pre-established, or fixed) or dynamic (e.g., created or modified at the time of execution).

A computer program product can include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium can include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like. A non-volatile computer-readable storage medium can also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium can also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium can also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium can include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RI IM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media can be substituted for or used in addition to the computer-readable storage media described above.

As should be appreciated, various embodiments of the present invention can also be implemented as methods, apparatus, systems, computing devices, computing entities, and/or the like. As such, embodiments of the present invention can take the form of an apparatus, system, computing device, computing entity, and/or the like executing instructions stored on a computer-readable storage medium to perform certain steps or operations. Thus, embodiments of the present invention can also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations can be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code can be performed sequentially such that one instruction is retrieved, loaded, and executed at a time. In some exemplary embodiments, retrieval, loading, and/or execution can be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

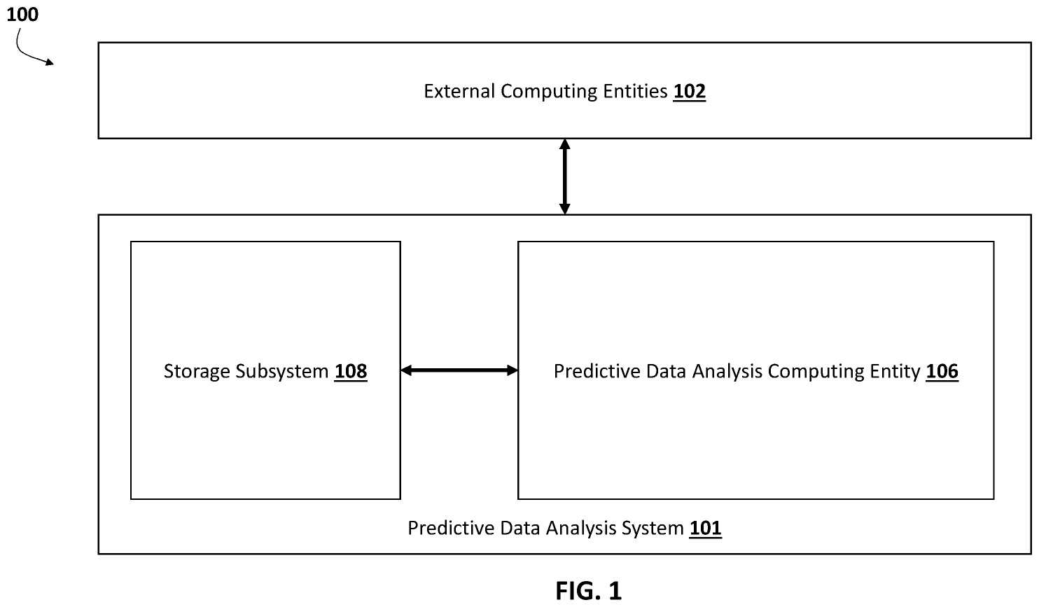

FIG. 1 is a schematic diagram of an example architecture 100 for performing predictive data analysis. The architecture 100 includes a predictive data analysis system 101 configured to receive predictive data analysis requests from external computing entities 102, process the predictive data analysis requests to generate predictions, provide the generated predictions to the external computing entities 102, and automatically perform prediction-based actions based at least in part on the generated predictions.

In some embodiments, predictive data analysis system 101 can communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 can include a predictive data analysis computing entity 106 and a storage subsystem 108. The predictive data analysis computing entity 106 can be configured to receive predictive data analysis requests from one or more external computing entities 102, process the predictive data analysis requests to generate predictions corresponding to the predictive data analysis requests, provide the generated predictions to the external computing entities 102, and automatically perform prediction-based actions based at least in part on the generated predictions.

The storage subsystem 108 can be configured to store input data used by the predictive data analysis computing entity 106 to perform predictive data analysis as well as model definition data used by the predictive data analysis computing entity 106 to perform various predictive data analysis tasks. The storage subsystem 108 can include one or more storage units, such as multiple distributed storage units that are connected through a computer network. Each storage unit in the storage subsystem 108 can store at least one of one or more data assets and/or one or more data about the computed properties of one or more data assets. Moreover, each storage unit in the storage subsystem 108 can include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

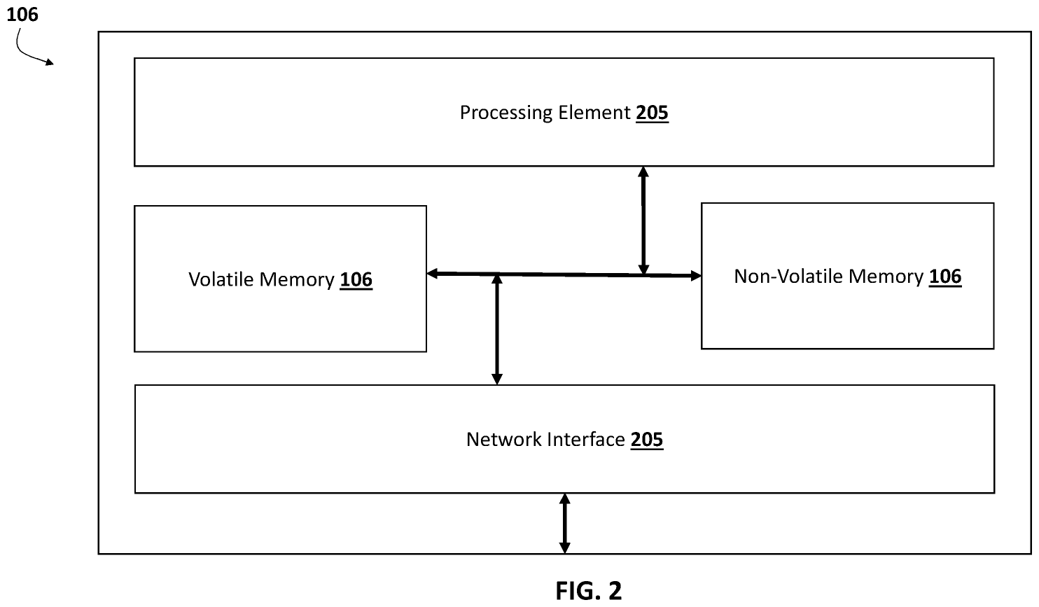

FIG. 2 provides a schematic of a predictive data analysis computing entity 106 according to one embodiment of the present invention. In general, the terms computing entity, computer, entity, device, system, and/or similar words used herein interchangeably can refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes can include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 can also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 can include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 can be embodied in a number of different ways.

For example, the processing element 205 can be embodied as one or more complex programmable logic devices (CPLDs), microprocessors, multi-core processors, coprocessing entities, application-specific instruction-set processors (ASIPs), microcontrollers, and/or controllers. Further, the processing element 205 can be embodied as one or more other processing devices or circuitry. The term circuitry can refer to an entirely hardware embodiment or a combination of hardware and computer program products. Thus, the processing element 205 can be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 can be configured for a particular use or configured to execute instructions stored in volatile or non-volatile media or otherwise accessible to the processing element 205. As such, whether configured by hardware or computer program products, or by a combination thereof, the processing element 205 can be capable of performing steps or operations according to embodiments of the present invention when configured accordingly.

In one embodiment, the predictive data analysis computing entity 106 can further include, or be in communication with, non-volatile media (also referred to as non-volatile storage, memory, memory storage, memory circuitry and/or similar terms used herein interchangeably). In one embodiment, the non-volatile storage or memory can include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media can store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably can refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 can further include, or be in communication with, volatile media (also referred to as volatile storage memory, memory storage, memory circuitry and/or similar terms used herein interchangeably). In one embodiment, the volatile storage or memory can also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media can be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like can be used to control certain aspects of the operation of the predictive data analysis computing entity 106 with the assistance of the processing element 205 and operating system.

As indicated, in one embodiment, the predictive data analysis computing entity 106 can also include one or more communications interfaces 220 for communicating with various computing entities, such as by communicating data, content, information, and/or similar terms used herein interchangeably that can be transmitted, received, operated on, processed, displayed, stored, and/or the like. Such communication can be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 can be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 can include, or be in communication with, one or more input elements, such as a keyboard input, a mouse input, a touch screen/display input, motion input, movement input, audio input, pointing device input, joystick input, keypad input, and/or the like. The predictive data analysis computing entity 106 can also include, or be in communication with, one or more output elements (not shown), such as audio output, video output, screen/display output, motion output, movement output, and/or the like.

### Exemplary External Computing Entity

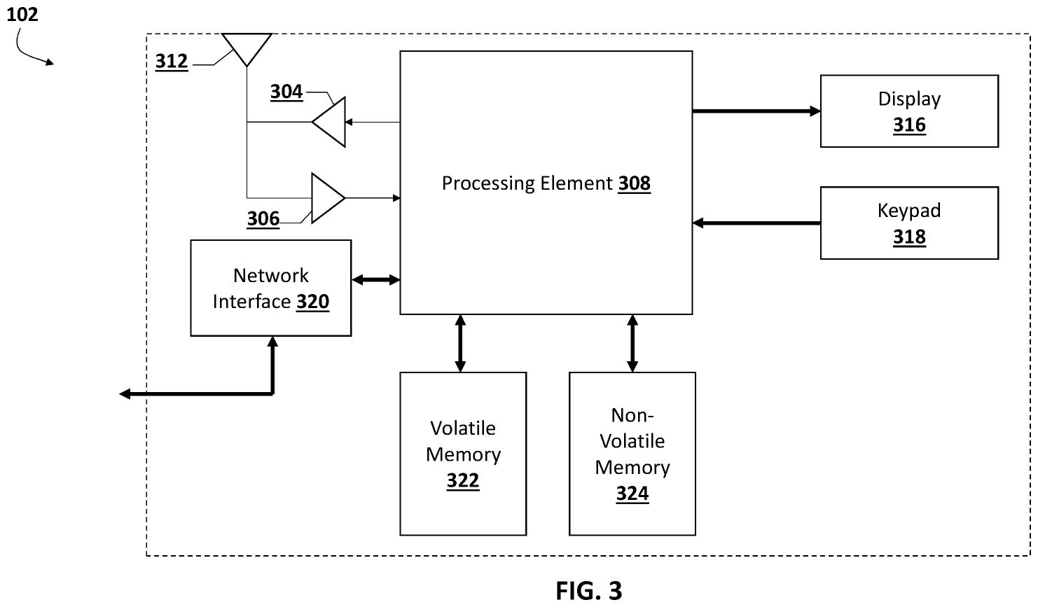

FIG. 3 provides an illustrative schematic representative of an external computing entity 102 that can be used in conjunction with embodiments of the present invention. In general, the terms device, system, computing entity, entity, and/or similar words used herein interchangeably can refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, can include signaling information/data in accordance with air interface standards of applicable wireless systems. In this regard, the external computing entity 102 can be capable of operating with one or more air interface standards, communication protocols, modulation types, and access types. More particularly, the external computing entity 102 can operate in accordance with any of a number of wireless communication standards and protocols, such as those described above with regard to the predictive data analysis computing entity 106. In a particular embodiment, the external computing entity 102 can operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 can operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 can include location determining aspects, devices, modules, functionalities, and/or similar words used herein interchangeably. For example, the external computing entity 102 can include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites can be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like.

Alternatively, the location information/data can be determined by triangulating the external computing entity's 102 position in connection with a variety of other systems, including cellular towers, Wi-Fi access points, and/or the like. Similarly, the external computing entity 102 can include indoor positioning aspects, such as a location module adapted to acquire, for example, latitude, longitude, altitude, geocode, course, direction, heading, speed, time, date, and/or various other information/data. Some of the indoor systems can use various position or location technologies including RFID tags, indoor beacons or transmitters, Wi-Fi access points, cellular towers, nearby computing devices (e.g., smartphones, laptops) and/or the like. For instance, such technologies can include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 can also comprise a user interface (that can include a display 316 coupled to a processing element 308) and/or a user input interface (coupled to a processing element 308). For example, the user interface can be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and can include a full set of alphabetic keys or set of keys that can be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or can be removable. For example, the non-volatile memory can be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The volatile memory can be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and nonvolatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this can include a user application that is resident on the entity or accessible through a browser or other user interface for communicating with the predictive data analysis computing entity 106 and/or various other computing entities.

In another embodiment, the external computing entity 102 can include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the external computing entity 102 can be embodied as an artificial intelligence (AI) computing entity, such as an Amazon Echo, Amazon Echo Dot, Amazon Show, Google Home, and/or the like. Accordingly, the external computing entity 102 can be configured to provide and/or receive information/data from a user via an input/output mechanism, such as a display, a camera, a speaker, a voice-activated input, and/or the like. In certain embodiments, an AI computing entity can comprise one or more predefined and executable program algorithms stored within an onboard memory storage module, and/or accessible over a network. In various embodiments, the AI computing entity can be configured to retrieve and/or execute one or more of the predefined program algorithms upon the occurrence of a predefined trigger event.

### V. Exemplary System Operations

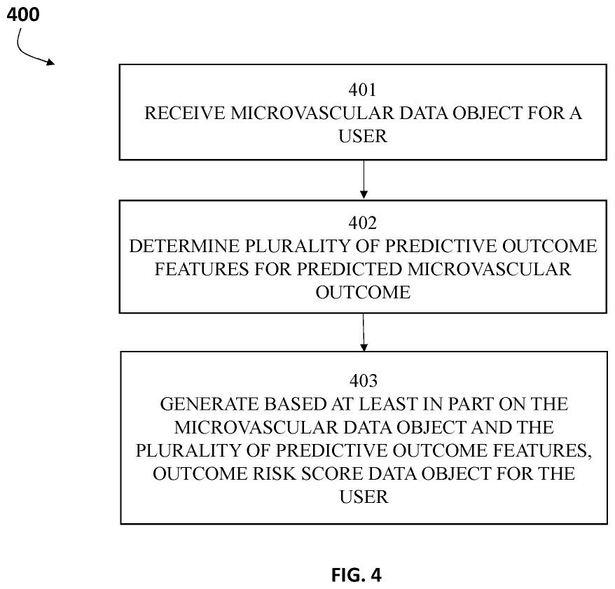

FIG. 4 is a flowchart diagram of an example process 400 for generating an outcome risk score data object for a predicted microvascular outcome in accordance with some embodiments discussed herein. Via the various steps/operations of the process 400, the predictive data analysis computing entity 106 can generate an outcome risk score data object for a predicted microvascular outcome using microvascular information. Unlike outputs from conventional health assessment techniques, the outcome risk score data object described herein can be reliable and accurate for short-term time intervals (e.g., one- to three-years, etc.) and can be dynamically adjusted over time based at least in part on real-world data. By utilizing microvascular information, the example process 400 can provide a technical improvement over conventional risk assessment techniques that are not capable of reliably or accurately predicting predicted microvascular outcomes and are limited to long-term time intervals (e.g., five or more years, etc.).

At step/operation 401, the process 400 can include receiving, by one or more processors, a microvascular data object for a user. For example, the predictive data analysis computing entity 106 can receive the microvascular data object for the user. The microvascular data object, for example, can be indicative of a microvascular volume differential for the user. The microvascular volume differential can represent one or more flow abnormalities in one or more regions of the user's body. In some embodiments, the microvascular volume differential for the user can be based at least in part on oxygen saturation levels in one or more different microvascular regions of the user's body. A microvascular volume differential can be indicative of a presence and/or severity of PAD including, for example, LE-PAD. PAD and LE-PAD, for instance, can be identified by one or more flow abnormalities at one or more different microvascular regions of the user's body.

The microvascular data object can include one or more microvascular health measurements. A microvascular health measurement can include a microvascular health value for the user that represents the user's microvascular blood flow. The microvascular health value can be represented by a percentage, fraction, and/or any other numerical representation for representing a proportion of the user's microvascular blood flow. As examples, a microvascular health value of 0.9 (e.g., 90%, 9/10, etc.) can indicate a high (e.g., healthy) blood flow rate for the user, whereas a microvascular health value of 0.4 (e.g., 40%, 4/10, etc.) can indicate a low (e.g., unhealthy) blood flow rate for the user.

The microvascular data object can be associated with one or more volume-based measurement techniques. The one or more volume-based measurement techniques can include VP techniques such as, for example, a PAD screen. By way of example, the microvascular data object can include a microvascular health value measured using one or more VP techniques. The VP techniques, for example, can obtain microvascular health measurements using VP systems for measuring microvascular blood flow volumes at one or more microvascular regions of the user. The VP techniques can include any number of different VP systems capable of measuring microvascular flow volumes at one or more different microvascular regions of the user's body. One exemplary VP system, for example, can include a peripheral artery disease screen such as, for example, QuantaFlo™ in which a sensor is placed on the user's left toe, right toe, left finger, and right finger for a period of time (e.g., fifteen seconds) to determine blood flow in each of the user's legs and arms. The blood flow data can be analyzed to determine a microvascular health measurement that is indicative of microvascular blood pressure between upper and lower extremities of a user.

At step/operation 402, the process 400 can include determining, by the one or more processors, a plurality of predictive outcome features for a predicted microvascular outcome. For example, the predictive data analysis computing entity 106 can determine the plurality of predictive outcome features for the predicted microvascular outcome. The plurality of predictive outcome features for the predicted microvascular outcome can include a plurality of general risk factors that are applicable to a plurality of different predicted microvascular outcomes. The plurality of risk factors, for example, can include smoking, lack of exercise, diet, obesity, high blood pressure, high cholesterol, medical history, age, and/or any other characteristic with an associative relationship to predicted microvascular outcomes.

In some embodiments, the plurality of predictive outcome features can be specific to a predicted microvascular outcome of interest. For example, the process 400 can include selecting a predicted microvascular outcome of interest. The predicted microvascular outcome of interest can include a specific microvascular disease such as, for example, asymptomatic LE-PAD, peripheral polyneuropathy, chronic kidney disease (CKD). In addition, or alternatively, the predicted microvascular outcome of interest can include a specific microvascular event such as, for example, MACE and mortality at 1- and 3-years post-screening. By way of example, the predicted microvascular outcome of interest can include MACE and mortality for a particular user. The plurality of predictive outcome features, for example, can include one or more characteristics with an associative or causative relationship with the predicted microvascular outcome of interest.

The plurality of predictive outcome features can be received from a plurality of different clinical sources. By way of example, clinical literature and/or expertise can be leveraged to source a list of clinically relevant predictive outcome features for predicted microvascular outcomes such as the predicted microvascular outcome of interest. In some embodiments, the plurality of predictive outcome features can be augmented by secondary features that may contribute to the likelihood of a predicted microvascular outcome. Secondary features, for example, can include social determinants of health as identified via deprivation index, etc. By way of example, areas of high deprivation can lead to increased risk for predicted microvascular outcomes.

In some embodiments, step/operation 402 can be performed responsive to a determination that a microvascular data object achieves a risk criteria.

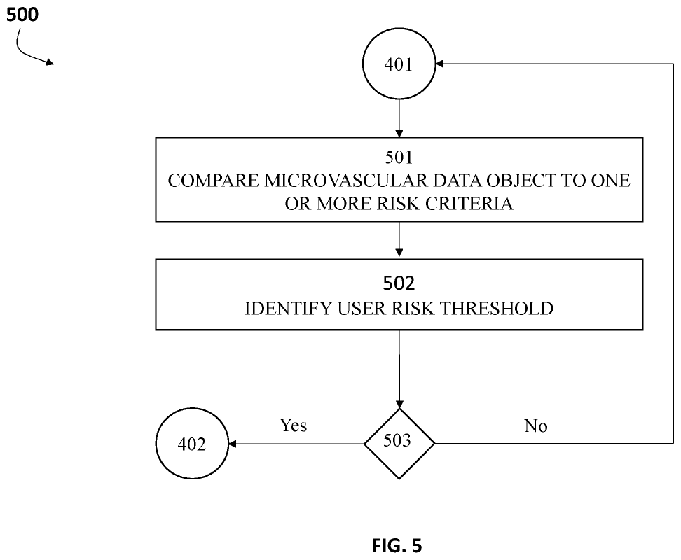

FIG. 5 is a flowchart diagram of an example process 500 for determining that the microvascular data object achieves risk criteria in accordance with some embodiments discussed herein. Via the various steps/operations of the process 500, the predictive data analysis computing entity 106 can preprocess a microvascular data object to determine whether a user satisfies one or more risk criteria. In some embodiments, the process 500 can include a plurality of operations subsequent to operation 401 of FIG. 4, where the process 400 includes receiving a microvascular data object for a user.

At step/operation 501, the process 500 can include comparing the microvascular data object to one or more risk criteria. For example, the predictive data analysis computing entity 106 can compare the microvascular data object to one or more risk criteria. The one or more risk criteria, for example, can include one or more outcome thresholds such as, for example, PAD thresholds.

The PAD thresholds can identify one or more thresholds and/or ranges of a microvascular health value that can be associated with different severities of PAD. For example, a first PAD threshold (e.g., 0.9 value) can indicate that a user is not at risk of PAD. If the user's microvascular data object is representative of a microvascular health value that achieves and/or exceeds the first PAD threshold, the predictive data analysis computing entity 106 can determine that the risk criteria is not satisfied. As another example, a second PAD threshold (e.g., 0.89 value) can indicate that a user is at risk of PAD. If the user's microvascular data object is representative of a microvascular health value that does not achieve or exceed the second PAD threshold, the predictive data analysis computing entity 106 can determine that the risk criteria is satisfied. As other examples, a first PAD range (e.g., 0.6 to 0.89 value) can indicate that a user has a low severity level of PAD; another, second PAD range (e.g., 0.4 to 0.6 value) can indicate that a user has an intermediate severity level of PAD; and yet another, third PAD range (e.g., 0.1 to 0.4 flow level) can indicate that a user has a high severity level of PAD.

The example PAD thresholds and ranges are provided herein for exemplary purposes. A person of ordinary skill in the art would understand that any number of PAD thresholds and ranges can be implemented depending on the application.

At step/operation 502, the process 500 can include identifying a user risk threshold for the user based at least in part on the comparison between the microvascular data object and the one or more risk criteria. For example, the predictive data analysis computing entity 106 can identify the user risk threshold for the user based at least in part on the comparison between the microvascular data object and the one or more risk criteria.

By way of example, the predictive data analysis computing entity 106 can compare a microvascular health value measured for the user to the one or more risk criteria to identify a risk threshold corresponding to the user. As one example, the predictive data analysis computing entity 106 can identify a risk threshold corresponding to the user by identifying a PAD threshold and/or range achieved by the microvascular health value. The identified user risk threshold, for example, can be indicative of a presence and/or severity of PAD for the user.

At step/operation 503, the process 500 can include determining whether the user risk threshold is outcome significant. For example, the predictive data analysis computing entity 106 can determine whether the user risk threshold is outcome significant. As described herein, the presence and/or severity of PAD for a user can be an independent predictor of one or more different predicted microvascular outcomes and, therefore, be outcome significant.

In the event that the user risk threshold satisfies one or more risk criteria indicative of the presence of PAD, the process 500 can determine that the user risk threshold is outcome significant and proceed to step/operation 402 of FIG. 4 in which the process 400 includes receiving a plurality of predictive outcome features for a predicted microvascular outcome. In this manner, an outcome risk score data object for the user can be generated in response to a determination that the microvascular data object does not achieve a threshold volume differential.

In the event that the user risk threshold does not satisfy one or more risk criteria indicative of the presence of PAD (e.g., indicating an absence of PAD), the process 500 can return to step/operation 401 in which the process 400 includes receiving a microvascular data object for another user.

Returning to FIG. 4, at step/operation 403, the process 400 can include generating an outcome risk score data object for the user. For example, the predictive data analysis computing entity 106 can generate, based at least in part on the microvascular data object and the plurality of predictive outcome features for the predicted microvascular outcome, the outcome risk score data object for the user.

The outcome risk score data object can be configured to describe the user's risk for the predicted microvascular outcome within a time period. For example, the outcome risk score data object for the user can be indicative of a short-term likelihood (e.g., one-to-three-year time span) that the user will develop a microvascular disease and/or a short-term likelihood (e.g., one-to-three-year time span) of an occurrence of a microvascular event. In addition, or alternatively, the outcome risk score data object for the user can be indicative of a long-term likelihood (e.g., five-year or longer time span) that the user will develop a microvascular disease and/or a long-term likelihood (e.g., e.g., five-year or longer time span) of an occurrence of a microvascular event.

The outcome risk score data object can be descriptive of an overall outcome risk score and/or a specific outcome risk score. For example, the overall outcome risk score can include the user's overall risk for any predicted microvascular outcome. The overall outcome risk score can be descriptive of the user's overall health. The specific outcome risk score can refer to the user's specific risk for a selected predicted microvascular outcome of interest. As described herein, a microvascular health value represented by the microvascular data object can be representative of the user's overall health. In some embodiments, the outcome risk score data object can include the microvascular health value. In addition, or alternatively, the microvascular health value can be used as a proxy for the overall outcome risk score.

In some embodiments, the microvascular health value can be refined using the plurality of predictive outcome features.

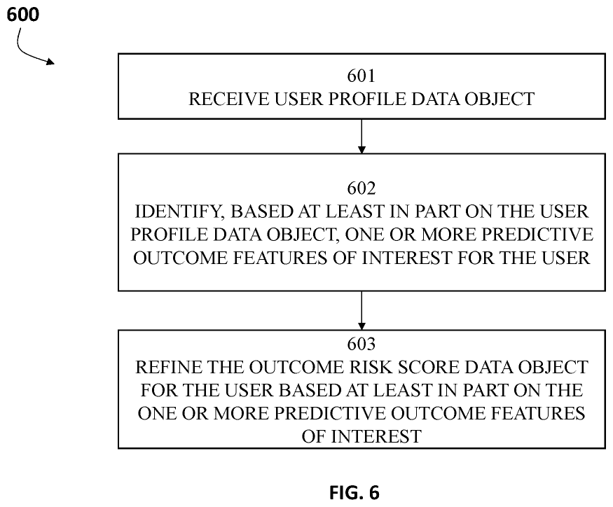

FIG. 6 is a flowchart diagram of an example process 600 for refining an outcome risk score for a user. Via the various steps/operations of the process 600, the predictive data analysis computing entity 106 can collect, analyze, and store the historical microvascular data object in an efficient and reliable manner. In some embodiments, the process 600 can include a plurality of operations subsequent to operation 403 of FIG. 4, where the process 400 includes generating, based at least in part on the microvascular data object and the plurality of predictive outcome features for the predicted microvascular outcome, an outcome risk score data object for the user. In addition, or alternatively, the process 600 can include one or more suboperations of operation 403 of FIG. 4.

At step/operation 601, the process 600 can include receiving a user profile data object for the user. For example, the predictive data analysis computing entity 106 can receive the user profile data object that is indicative of user information (e.g., user preferences) for the user.

A user can include an individual person such as a patient, an employee, and/or the like. The user information can include user characteristics, associated predictive outcome features, microvascular health measurements, and/or any other information for the determination and/or mitigation of predicted microvascular outcomes for the particular user.

The user profile data object can describe one or more user characteristics for a user including, as examples, a user medical history (e.g., allergies, historical clinical procedures/recommendations, etc.), demographic information (e.g., social determinants, etc.), preferences (e.g., habits such as smoking, diagnosis preferences, etc.), and/or the like.

As another example, the user profile data object can describe a historical and/or current overall health for the particular user. A user's overall health, for example, can be represented by a microvascular health measurement for the user. The user profile data object can describe a plurality of historical and/or current microvascular health measurements for the user. In some embodiments, the user profile data object can include an analysis of one or more trends and/or other secondary insights associated with the historical and/or current microvascular health measurements for the user.

As yet another example, the user profile data object can describe one or more predictive outcome features associated with the user. In some embodiments, the one or more predictive outcome features described by the user profile data object can correspond to user characteristics for the user. By way of example, the associated predictive outcome features can include historical clinical procedures, social determinants, smoking habits and/or any other characteristics of a particular user.

At step/operation 602, the process 600 can include identifying, based at least in part on the user profile data object, one or more predictive outcome features of interest for the user. For example, the predictive data analysis computing entity 106 can identify, based at least in part on the user profile data object, the one or more predictive outcome features of interest from the plurality of predictive outcome features. By way of example, in some embodiments, one or more predictive outcome features of the plurality of predictive outcome features can correspond to user characteristics for the user. For instance, the associated predictive outcome features can include historical clinical procedures, social determinants, smoking habits, and/or any other characteristics of the user that can impact and/or be associated with a likelihood of a predicted microvascular outcome.

At step/operation 603, the process 600 can include refining the outcome risk score data object for the user based at least in part on the one or more predictive outcome features of interest. For example, the predictive data analysis computing entity 106 can refine the outcome risk score data object for the user based at least in part on the one or more predictive outcome features of interest.

For instance, the microvascular health value can be refined using the plurality of predictive outcome features. The resulting outcome risk score data object for the user can include an integrated risk evaluation for predicted microvascular outcomes that integrates (i) a direct measure of overall health (e.g., microvascular health value) with (ii) associative features such as, for example, one or more predictive outcome features that can have an association with predicted microvascular outcomes and (iii) causative features such as, for example, one or more predictive outcome features that can have a causal impact on predicted microvascular outcomes.

An association or causal impact of a respective predictive outcome feature to an outcome risk score data object and, consequently, a predicted microvascular outcome, can be determined based at least in part on a correlation between the respective predictive outcome feature and a historical microvascular data object. By way of example, a microvascular health value can be utilized as a proxy for an outcome risk score at a certain period of time. Fluctuations between historical microvascular health values measured over time can be correlated with a respective predictive outcome feature to determine a causal impact of the respective predictive outcome feature on the outcome risk score data object and, consequently, the predicted microvascular outcome.

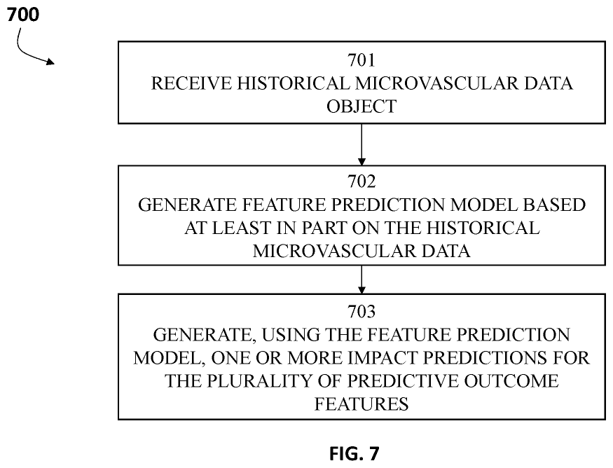

FIG. 7 is a flowchart diagram of an example process 700 for generating an outcome risk score data object for a user based at least in part on a historical microvascular data object. Via the various steps/operations of the process 700, the predictive data analysis computing entity 106 can collect, analyze, and store a historical microvascular data object in an efficient and reliable manner. In some embodiments, the process 700 can include a plurality of operations preceding operations 403 of FIGS. 4 and 603 of FIG. 6, where the processes 400 and 600 include generating an outcome risk score data object for a user.

At step/operation 701, the process 700 can include receiving a historical microvascular data object. For example, the predictive data analysis computing entity 106 can receive the historical microvascular data object for a plurality of users.

The historical microvascular data object can describe a plurality of historical microvascular health measurements for a plurality of users over time. For example, the plurality of historical microvascular health measurements for the plurality of users over time can include a plurality of microvascular health values computed for each of the plurality of users. The plurality of historical microvascular health measurements can be measured during one or more historical clinical visits, house calls, etc. in which a user's microvascular health can be observed. In some embodiments, volume-based measurement techniques can be used by a user to self-diagnosis the user's microvascular health. The historical microvascular data object can include a historical log of a plurality of historical microvascular health measurements for each of the plurality of users. Each historical microvascular health measurement can be recorded and associated with a particular user at a particular time. In some embodiments, the plurality of historical microvascular health measurements can be stored in association with a user profile.

In some embodiments, the process 700 can include receiving historical user data and/or third-party data associated with the historical microvascular data object. The historical user data and/or third-party data can include user information (e.g., logged in a user profile data object, etc.) and/or third-party analysis (e.g., third-party statistics, research, etc.) associated with historical microvascular health measurements. The historical user data and/or third-party data can be received by following volume-based measurement techniques over time and correlating each historical microvascular health measurement with contextual information associated with the volume-based measurement technique. The contextual information, for example, can identify a timing of the volume-based measurement technique, a user associated with the volume-based measurement technique, third-party analysis for the volume-based measurement technique, etc.

At step/operation 702, the process 700 can include generating a feature prediction model based at least in part on the historical microvascular data object. For example, the predictive data analysis computing entity 106 can generate the feature prediction model based at least in part on the historical microvascular data object. The feature prediction model can correlate the historical microvascular data object and the plurality of predictive outcome features over time. For instance, the feature prediction model can be configured to predict one or more impact predictions for the plurality of predictive outcome features based at least in part on one or more mapped relationships between the historical microvascular data object and the plurality of predictive outcome features over time.

As one example, the feature prediction model can include a time-series model that is descriptive of a plurality of historical microvascular health measurements and a plurality of predictive outcome features associated with each of the plurality of historical microvascular health measurements over time. The time-series model can be generated by tabulating data sources for predictive modeling of the plurality of predictive outcome features against predicted microvascular outcomes. The information can be collated in a time series format versus predicted microvascular outcomes for the plurality of users. The time series format can utilize any time series framework that can be applied for modeling purposes. In this manner, a feature prediction model can be generated that includes a plurality of microvascular health measurements that are recorded as a function of time in correlation with one or more predictive outcome features over time. By way of example, the feature prediction model can employ social determinants of health as one component, a direct measure of microvasculature as another component, a clinical measure as a third, etc.

As another example, the feature prediction model can include a machine-learning based model. The feature prediction model can include any type of machine-learning based model including one or more supervised, unsupervised, and/or reinforcement learning models. In some implementations, the feature prediction model can include a first machine-learning based prediction model configured to learn one or more associations between the plurality of predictive outcome features and a predicted microvascular outcome based at least in part on microvascular information. By way of example, the feature prediction model can include one or more neural networks (e.g., feedforward artificial neural networks, perceptron and multilayer perceptron neural networks, radial basis function artificial neural networks, recurrent neural networks, modular neural networks, etc.) trained using one or more supervised, unsupervised, and/or reinforcement training techniques based at least in part on a historical microvascular data object.

At step/operation 703, the process 700 can include generating, using the feature prediction model, one or more impact predictions for the plurality of predictive outcome features. For example, the predictive data analysis computing entity 106 can generate, using the feature prediction model, the one or more impact predictions for the plurality of predictive outcome features. The one or more impact predictions for the plurality of predictive outcome features can be indicative of a potential impact of a predictive outcome feature for the predicted microvascular outcome on the outcome risk score data object. The impact of a respective predictive outcome feature on the predicted microvascular outcome can be based at least in part on one or more fluctuations to microvascular health measurements associated with the respective predictive outcome feature.

For example, predictive modeling can be applied to predict how various predictive outcome features impact microvascular health measurements for a plurality of users over time. As described herein, microvascular health measurements can be utilized as a direct measure of overall health for a user. In this manner, inferences can be made regarding the impact of a predictive outcome feature on a predicted microvascular outcome (and/or risk thereof) based at least in part on predictive outcome features that impact variation of microvascular health measurements through time, both positively and negatively. For example, the presence of a respective predictive outcome feature such as, for example, a smoking habit during a time period between two progressively negative microvascular health measurements can be used to infer that the predictive outcome feature may have a negative causal impact on a predicted microvascular outcome. As another example, the presence of a predictive outcome feature such as, for example, an exercise habit during a time period between two progressively positive microvascular health measurements can be used to infer that the predictive outcome feature may have a positive causal impact on a predicted microvascular outcome.

In this manner, by adding microvascular health measurements to other potential predictive outcome features, the systems and methods of the present disclosure affords the opportunity to develop a new, more useful, integrated outcome risk score capable of accounting for and measuring the associative and/or causal properties of potential predictive outcome features. This risk score can subsequently be leveraged as part of a remedial support tool to guide shared decision making between users toward optimal therapeutic management to mitigate health risks for users.

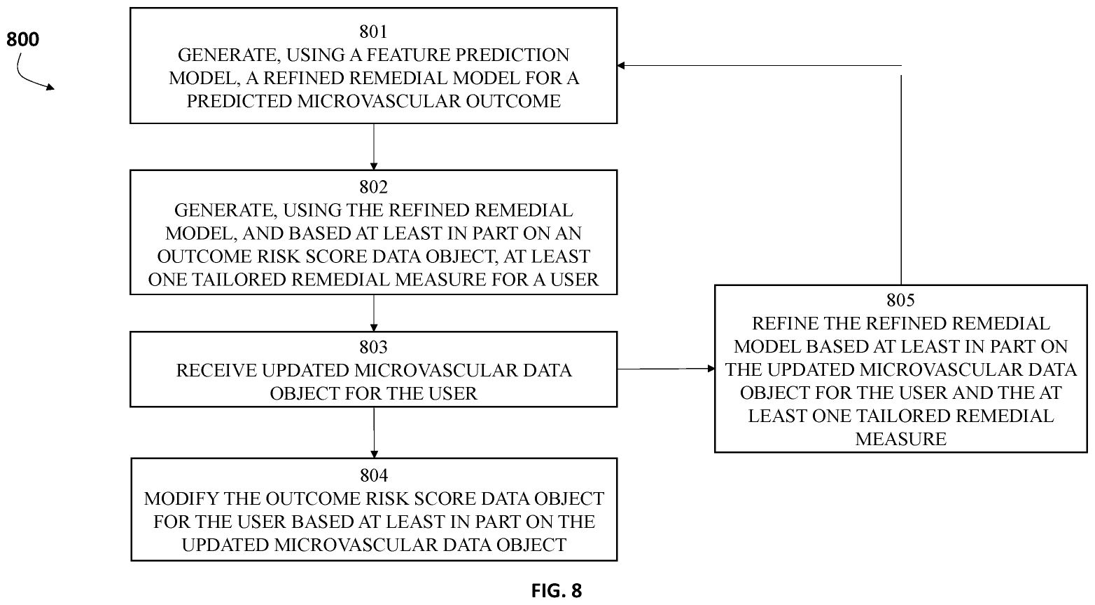

FIG. 8 is a flowchart diagram of an example process 800 for generating a tailored remedial measure for a user based at least in part on a microvascular data object. Via the various steps/operations of the process 800, the predictive data analysis computing entity 106 can generate a real-time tailored remedial measure for a user based at least in part on real-time information using a refined remedial model. In some embodiments, the process 800 can include a plurality of operations subsequent to operation 403 of FIG. 4, where the process 400 includes generating, based at least in part on the microvascular data object and the plurality of predictive outcome features for the predicted microvascular outcome of interest, an outcome risk score data object for the user.

As described herein, the systems and methods of the present disclosure enable the direct measurement of overall health using microvascular health measurements to determine associative and causative features for a predicted microvascular outcome. This information can be leveraged to identify causal features (e.g., predictive outcome features that have a causal impact on a predicted microvascular outcome) that can potentially be modified through remedial measures.

At step/operation 801, the process 800 can include generating a refined remedial model for a predicted microvascular outcome. For example, the predictive data analysis computing entity 106 can generate the refined remedial model for the predicted microvascular outcome using a feature prediction model. The refined remedial model, for example, can quantify a potential impact of at least one of the plurality of predictive outcome features on an outcome risk score data object. By way of example, the refined remedial model can include a causal propensity model configured to quantify statistically the impact of predictive outcome features upon real-world change (e.g., as represented by microvascular health measurements).

As another example, the refined remedial model can include a machine-learning based model. The refined remedial model can include any type of machine-learning based model including one or more supervised, unsupervised, and/or reinforcement learning models. In some implementations, the refined remedial model can include a second machine-learning based prediction model configured to learn one or more associations between the plurality of predictive outcome features and a microvascular data object for a user based at least in part on microvascular information for a plurality of users. By way of example, the refined remedial model can include one or more neural networks (e.g., feedforward artificial neural networks, perceptron and multilayer perceptron neural networks, radial basis function artificial neural networks, recurrent neural networks, modular neural networks, etc.) trained using one or more supervised, unsupervised, and/or reinforcement training techniques based at least in part on a historical microvascular data object.

At step/operation 802, the process 800 can include generating a tailored remedial measure data object for a user using the refined remedial model. For example, the predictive data analysis computing entity 106 can generate the tailored remedial measure data object for the user using the refined remedial model. The tailored remedial measure data object for the user can describe a remedial measure for a user to mitigate a predicted microvascular outcome.

The remedial measure can be based at least in part on the microvascular data object and the outcome risk score data object for the user. The tailored remedial measure can be associated with at least one of the plurality of predictive outcome features. For example, in response to a negative outcome risk score data object and a corresponding microvascular data object, the tailored remedial measure can identify one or more predictive outcome features associated with the user that may be modified through treatments or altered behavior.

The tailored remedial measure data object can include a plurality of alternative remedial measures and an indication of an expected impact to the outcome risk score associated with at least one of the plurality of alternative remedial measures. For example, using the refined remedial model, the tailored remedial measure data object can provide a plurality of different options for managing an outcome risk score data object. Each option can identify (i) a treatment and/or altered behavior associated with a predictive outcome feature and (ii) a potential impact to the outcome risk score data object based at least in part on the treatment and/or altered behavior. As a consequence, the tailored remedial measure data object can include a “next-best option” for a given user in the event one option is preferred over another (e.g., a user refuses to quit smoking, but an increase to their statin results in similar risk mitigation).

In some implementations, the tailored remedial measure data object can be based at least in part on user data. For example, the predictive data analysis computing entity 106 can receive a user profile data object descriptive of user information for the user. As one example, the user profile data object can include one or more user preferences. In some implementations, the plurality of different options for managing the outcome risk score data object can be selected, ordered, and/or presented based at least in part on one or more user preferences.

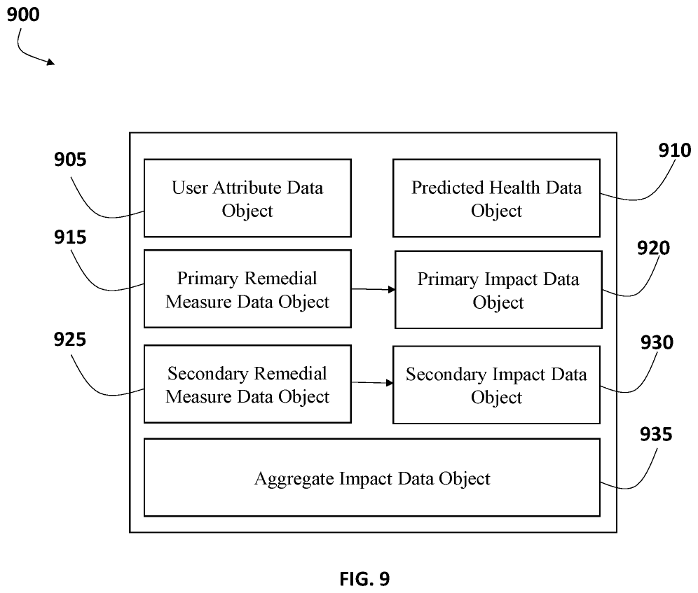

By way of example, FIG. 9 is an operational example of an interactive user interface 900 for selectively providing alternative treatments based at least in part on user preferences. The interactive user interface 900 can be implemented by a clinical decision engine configured to provide tailored remedial measures based at least in part on a user's overall health. The interactive user interface 900 can include a plurality of interactive widgets indicative of the at least one tailored remedial measure for the user and one or more alternative remedial measures for the user. By way of example, the plurality of interactive widgets can be indicative of a user attribute data object 905, predicted health data object 910, primary remedial measure data object 915, primary impact data object 920, secondary tailored remedial measure data object 925, a secondary impact data object 930 and/or an aggregate impact data object 935. Each of the data objects can be dynamically tailored to a particular user based at least in part on the user's current overall health and/or other user information.

For example, the user attribute data object 905 can include user information for a user. The user information, for example, can include user characteristics, associated predictive outcome features, microvascular health measurements, and/or any other information for the diagnosis and treatment of predicted microvascular outcomes for the user. The predicted health data object 910 can be represented by an outcome risk score data object for the user. In addition, or alternatively, in some implementations, the predicted health data object 910 can be represented by a current microvascular health measurement for the user as described herein.

The primary remedial measure data object 915 can represent a first tailored remedial measure (e.g., increased statins, quit smoking habit, etc.) for the user to mitigate an outcome risk score data object and/or improve a current microvascular health measurement for the user. The primary impact data object 920 can be indicative of an expected impact to the predicted health data object for the user in response to the first tailored remedial measure.

The secondary tailored remedial measure data object 925 can represent a second tailored remedial measure (e.g., increased statins, quit smoking habit, etc.) for the user to mitigate an outcome risk score data object and/or improve a current microvascular health measurement for the user. The secondary impact data object 930 can be indicative of an expected impact to the predicted health data object for the user in response to the second tailored remedial measure.

The aggregate impact data object 935 can be indicative of an expected impact to the predicted health data object 910 for the user in response to both the first and second tailored remedial measures.

The primary impact data object 920, the secondary impact data object 930, and the aggregate impact data object 935 can be determined based at least in part on historical correlations between a predictive outcome feature associated with the first and second tailored remedial measures and microvascular health measurements as described herein.

The predictive data analysis computing entity 106 can provide the interactive user interface 900 to the user. As described herein, the interactive user interface 900 for the user can include an indication of an expected impact (e.g., primary impact data object 920, the secondary impact data object 930, the aggregate impact data object 935) to the outcome risk score data object (e.g., predicted health data object 910) associated with the at least one tailored remedial measure (e.g., primary remedial measure data object 915) for the user and the one or more alternative remedial measures (e.g., secondary tailored remedial measure data object 925) for the user. The interactive user interface 900 for the user can also include an indication of the outcome risk score data object (e.g., predicted health data object 910) for the user. The predictive data analysis computing entity 106 can receive data indicative of a selection of at least one of the plurality of interactive widgets. Responsive to the selection of the at least one interactive widget, the predictive data analysis computing entity 106 can automatically update, using the refined remedial model, the outcome risk score data object (e.g., predicted health data object 910) for the user.

The predictive data analysis computing entity 106 can automatically track a user's overall health over a period of time. In this way, the interactive user interface 900 can be automatically reconfigured based at least in part on real world information representative of a particular user's overall health. Moreover, unlike traditional health risk assessment systems, the interactive user interface 900 can provide multiple treatment options as well as contextual information associated with each option. In this manner, the interactive user interface 900 includes an improved user interface in which treatment information tailored to a user is automatically displayed based at least in part on current microvascular conditions.

Turning back the FIG. 8, at step/operation 803, the process 800 can include receiving an updated microvascular data object for the user. For example, the predictive data analysis computing entity 106 can receive the updated microvascular data object for the user. The updated microvascular data object can be received at a measurement interaction (e.g., clinical visits, house calls, etc.) subsequent to the determination of the microvascular data object. For example, the overall health for a user can be monitored by conducting volume-based measurement techniques at a regular interval to repeatedly receive updated microvascular data objects.

In some implementations, the updated microvascular data object can be utilized to perform offline risk scoring, at regular time periods, across populations of users automatically. In this manner, updated microvascular data objects can be deployed at the operational level, in order to inform treatment interactions such as, for example, by prioritizing house calls to check on higher risk users.

At step/operation 804, the process 800 can include modifying the outcome risk score data object for the user. For example, the predictive data analysis computing entity 106 can modify the outcome risk score data object for the user based at least in part on the update microvascular data object. The predictive data analysis computing entity 106, for example, can modify the outcome risk score for the user based at least in part on the updated microvascular data by recomputing the outcome risk score using the updated microvascular data object as described herein.

At step/operation 805, the process 800 can include refining the refined remedial model based at least in part on the updated microvascular data object. For example, the predictive data analysis computing entity 106 can refine the refined remedial model based at least in part on the updated microvascular data object for the user and the at least one tailored remedial measure for the user. The refined remedial model, for example, can be continuously refined based at least in part on real world data. For instance, the refined remedial model can include a learnable model (e.g., causal propensity model, machine-learning based model such as a neural network, etc.) with a plurality of modifiable parameters based at least in part on changes over time. The refined remedial model can be improved over time by assessing, during future measuring interactions and repeated risk scoring, how a user's improvement changes over time. These changes can be incorporated as a feedback loop (e.g., concordant therapy) to refine the output of the refined remedial model based at least in part on real-world outcomes.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for predicting predicted microvascular outcomes using microvascular data objects, the computer-implemented method comprising:
receiving, by one or more processors, a microvascular data object for a user, the microvascular data object indicative of a microvascular volume differential for the user;
determining, by the one or more processors, a plurality of predictive outcome features for a predicted microvascular outcome;
determining, by the one or more processors and using a feature prediction model, one or more impact predictions for the plurality of predictive outcome features;
generating, by the one or more processors and based at least in part on the microvascular data object and the one or more impact predictions, an outcome risk score data object for the user; and
generating, by the one or more processors, using a refined remedial model and based at least in part on the outcome risk score data object, at least one tailored remedial measure for the user.

2. The computer-implemented method of claim 1, wherein the microvascular data object is associated with one or more volume-based measurement techniques.

3. The computer-implemented method of claim 2, wherein the one or more volume-based measurement techniques comprise a peripheral artery disease screen.

4. The computer-implemented method of claim 1, wherein the microvascular data object is indicative of a presence or severity of peripheral artery disease.

5. The computer-implemented method of claim 1, further comprising:
generating, by the one or more processors, the outcome risk score data object for the user in response to a determination that the microvascular data object does not achieve a threshold differential.

6. The computer-implemented method of claim 1, wherein the predicted microvascular outcome comprises a major adverse cardiovascular event.

7. The computer-implemented method of claim 1, wherein the outcome risk score data object for the user is indicative of a risk of an occurrence of the predicted microvascular outcome within a one-to-three-year time span.

8. The computer-implemented method of claim 1, wherein the one or more impact predictions for the plurality of predictive outcome features are indicative of a potential impact of a predictive outcome feature for the predicted microvascular outcome on the outcome risk score data object.

9. The computer-implemented method of claim 1, further comprising:
receiving, by the one or more processors, a historical microvascular data object for a plurality of users, wherein the historical microvascular data object is indicative of a plurality of microvascular data objects for the plurality of users; and
generating, by the one or more processors, the feature prediction model based at least in part on the historical microvascular data object, wherein the feature prediction model is configured to predict the one or more impact predictions for the plurality of predictive outcome features based at least in part on one or more mapped relationships between the historical microvascular data object and the plurality of predictive outcome features over time.

10. The computer-implemented method of claim 9, further comprising:
generating, by the one or more processors and using the feature prediction model, the refined remedial model, wherein the refined remedial model is continuously refined based at least in part on real world data.

11. The computer-implemented method of claim 10, further comprising:
receiving, by the one or more processors, an updated microvascular data object for the user; and
modifying, by the one or more processors, the outcome risk score data object for the user based at least in part on the updated microvascular data object.

12. The computer-implemented method of claim 11, further comprising:
refining, by the one or more processors, the refined remedial model based at least in part on the updated microvascular data object for the user and the at least one tailored remedial measure for the user.

13. The computer-implemented method of claim 1, wherein generating, using the refined remedial model and based at least in part on the outcome risk score data object, the at least one tailored remedial measure for the user, comprises:
receiving, by the one or more processors, a user profile data object for the user that is indicative of one or more user preferences for the user; and
generating, by the one or more processors, using the refined remedial model, and based at least in part on the one or more user preferences for the user, the at least one tailored remedial measure for the user.

14. An apparatus for predicting predicted microvascular outcomes using microvascular data objects, the apparatus comprising at least one processor and at least one memory including program code, the at least one memory and the program code configured to, with the processor, cause the apparatus to at least:
receive a microvascular data object for a user, the microvascular data object indicative of a microvascular volume differential for the user;
determine a plurality of predictive outcome features for a predicted microvascular outcome;
determine, using a feature prediction model, one or more impact predictions for the plurality of predictive outcome features;
generate, based at least in part on the microvascular data object and the one or more impact predictions, an outcome risk score data object for the user; and
generate, using a refined remedial model and based at least in part on the outcome risk score data object, at least one tailored remedial measure for the user.

15. The apparatus of claim 14, wherein the microvascular data object comprises a parameter representative of a microvascular blood flow for the user at a point in time.

16. The apparatus of claim 14, further caused to at least:
provide, based at least in part on the at least one tailored remedial measure for the user, an interactive user interface for the user, wherein the interactive user interface comprise a plurality of interactive widgets indicative of the at least one tailored remedial measure for the user and one or more alternative remedial measures for the user.

17. The apparatus of claim 16, wherein the interactive user interface for the user comprises an indication of an expected impact to the outcome risk score data object associated with the at least one tailored remedial measure for the user and the one or more alternative remedial measures for the user.

18. The apparatus of claim 16, wherein the interactive user interface for the user comprises an indication of the outcome risk score data object for the user.

19. The apparatus of claim 18, further caused to at least:
receive data indicative of a selection of at least one of the plurality of interactive widgets; and
responsive to the selection of the at least one interactive widget, automatically update, using the refined remedial model, the outcome risk score data object for the user.

20. A computer program product for predicting predicted microvascular outcomes using microvascular data objects, the computer program product comprising at least one non-transitory computer-readable storage medium having computer-readable program code portions stored therein, the computer-readable program code portions configured to:
receive, by one or more processors, a microvascular data object for a user, the microvascular data object indicative of a microvascular volume differential for the user;
determine, by one or more processors, a plurality of predictive outcome features for a predicted microvascular outcome;
determine, by the one or more processors and using a feature prediction model, one or more impact predictions for the plurality of predictive outcome features;
generate, by the one or more processors and based at least in part on the microvascular data object and the one or more impact predictions, an outcome risk score data object for the user; and
generate, by the one or more processors, using a refined remedial model and based at least in part on the outcome risk score data object, at least one tailored remedial measure for the user.
